(19) United States
(12) Patent Application Publication
(10) Pub. No.: US 2012/0183918 A1
(43) Pub. Date: Jul. 19, 2012

(54) APPARATUS FOR DETECTING INFECTED TISSUE

(75) Inventors: Ian THOMPSON, Essex (GB); Tim WATSON, Herts (GB); Richard COOK, Kent (GB)

(73) Assignee: OSspray Ltd.

(21) Appl. No.: 13/293,680

(22) Filed: Nov. 10, 2011

Related U.S. Application Data
(63) Continuation of application No. 12/306,086, filed on Jun. 16, 2009, now abandoned.

(30) Foreign Application Priority Data
Jun. 26, 2006 (GB) 0612638.7

(51) Int. Cl. A61B 1/24; A61C 3/00

(52) U.S. Cl. 433/27; 433/29; 433/215

(57) ABSTRACT

An apparatus and method for detecting infected tissue are disclosed. The apparatus comprises a light source for producing a light beam and an optical element having an input end optically coupled to the light source and an output end arranged to direct the light beam as a confocal beam into tissue. The apparatus also comprise a detector optically coupled to the optical element to receive a return beam back from the tissue stimulated by the confocal beam and to generate an output dependent upon the return beam.

The present invention relates to an apparatus for detecting infected tissue, and may be used for discriminating between infected and sound tissue in a tooth for example.

Dental caries is a bacterial degradation process that starts in the outer highly mineralised enamel and then spreads to the inner dentine. The dentine consists of a protein (collagen) surrounded by mineral. Bacterial metabolic products lead to demineralisation and protein breakdown within the tooth. Within the dentine the carious lesion consists of two main parts. The superficial ‘caries infected dentine’—that which is heavily loaded with a variety of bacterial organisms and the ‘caries affected dentine’—that which is partially demineralised and has altered mechanical properties, but which is otherwise mainly free of bacteria.

The treatment of a decayed tooth often involves the removal of the infected dentine. In dentistry there is a perennial problem of the detection of remaining infected decayed tooth material overlying sound but stained affected and structurally adequate residual tissue. In clinical terms this equates to indicating to a clinician when to stop drilling away stained dentine—as the tactile sensation received from a high speed dental drill is remarkably poor at showing the transition from unsound decayed dentine to stained (similar colour) but structurally adequate tissue for restorative purposes. Excessive drilling may lead to unnecessary removal of tooth tissue with consequential dental pain, pulpal trauma, pulp death and even eventual loss of the tooth. A device for discriminating between infected decayed tooth material and structurally adequate residual tissue has been long sought after. For example techniques using decay sensing dyes have been proposed and developed.

Quantitative Laser Fluorescence (QLF) and Diagnodent™ decay detecting instruments are available and sample the bulk of a tooth in situ. Such instruments have illumination and detection channels for the light wavelengths employed (often in the infra red). Such instruments look to detect the presence of bulk decay and give an indication in marginal cases of whether to drill or not.

Autofluoresence is the ability of a material to emit light of longer wavelength and lower energy when an unadulterated material is illuminated by light of a short wavelength. Dentine has an inherent autofluorescence signal (green wavelengths excited by blue˜450-490 nm) and carious infected dentine has a different inherent autofluoresence signal. When trying to detect a signal indicative of carious infected dentine, such a signal may often be missed due to “swamping” of the decayed signal by the overwhelming bulk fluorescence signal from the tooth. As bulk decay is removed during a filling procedure, so the infected material film thickness decreases and is therefore increasingly unlikely to be detected by the current optical instruments.

According to a first aspect of the present invention there is provided an apparatus for detecting infected tissue, the apparatus comprising

a light source for producing a light beam;

an optical element having an input end optically coupled to the light source and an output end arranged to direct the light beam as a confocal beam into tissue;

a detector optically coupled to the optical element to receive a return beam back from the tissue stimulated by the confocal beam and to generate an output dependent upon the return beam and

an analyser for analysing the output of the detector to determine whether the return beam is indicative of infected tissue.

The use of a confocal beam allows it to be directed to a specific predetermined point or depth in tissue, such as a tooth, thus eliminating the ‘swamping effect’ of the bulk background signals. Thus, such an apparatus is significantly more sensitive at detecting infected tissue. An embodiment of the present invention is thus able to indicate when to stop drilling a decayed portion of a tooth so that an excessive amount of sound tissue is not drilled away.

The inventors have developed and trialed an embodiment designed around the inherent confocal behaviour of a fine multi filament, fibre optic cable to produce an instrument with optical sectioning depths of approximately 400 microns in dry conditions. This may be considered adequate as a dental practitioner probably cannot drill to greater accuracy and this far exceeds the sectioning capabilities of any of the current caries detection instruments available.

Generally confocal beams imply the presence of an identical aperture in both illumination and detection light pathways of a microscopic imaging instrument. The apertures are placed at the Conjugate Focal plane. The effect is to generate an optical tomographic effect, minimising the optical section depth from which light is detected. Light from above and below the optical plane levels is discarded—thereby developing the plane of section. This can have a significant benefit in detection of shallow carious lesions as the background bulk/gross autofluorescence of the remaining tooth dentine is excluded from the assay and therefore cannot overwhelm that from the decayed dentine.

The apparatus may be arranged to vary the predetermined point or depth in the tissue at which the confocal beam is directed. This may be achieved with a suitable mechanism as is well known to those skilled in the art.

The analyser is preferably arranged to reduce the effect of ambient light. This may be achieved by subtracting an output indicative of just ambient light from an output indicative of both ambient light and a return beam from the tissue stimulated by the confocal beam.

A dental device may be controlled by an apparatus of the first aspect of the present invention. Examples of possible dental devices to be controlled by the apparatus of the first aspect of the present invention include dental hard tissue removal devices, rotary and hand instrumentation, air abrasion devices, laser ablation devices and chemical and biological hard tissue removal devices. Such a dental device may include an apparatus according to the first aspect of the present invention.

According to a second aspect of the present invention there is provided a method of detecting infected tissue, the method comprising

producing a light beam;

directing the light beam into an input end of an optical element having an output end arranged to direct the light beam as a confocal beam into tissue;

detecting a return beam back from the tissue stimulated by the confocal beam;

generating an output dependent upon the detected return beam and

analysing the generated output to determine whether the return beam is indicative of infected tissue.

Embodiments of the present invention will now be described, by way of example only, with reference to the accompanying drawings in which:

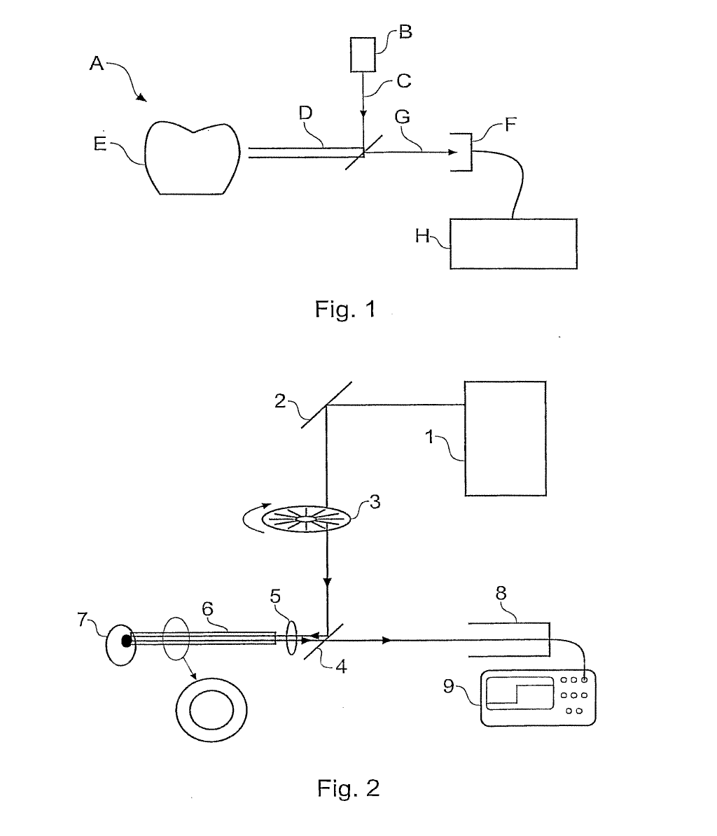

FIG. 1 shows a schematic diagram of a first embodiment of an apparatus for discriminating between infected and sound tissue;

FIG. 2 shows a schematic diagram of a second embodiment of an apparatus for discriminating between infected and sound tissue;

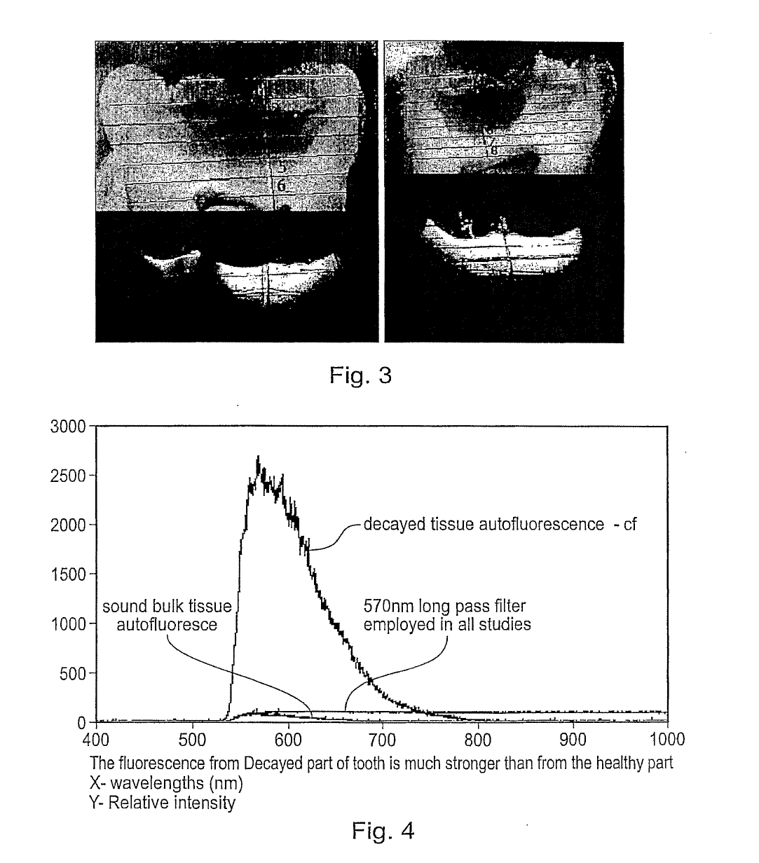

FIG. 3 shows images of two sectioned dentine decay lesions;

FIG. 4 shows a received fluorescence signal from a tooth;

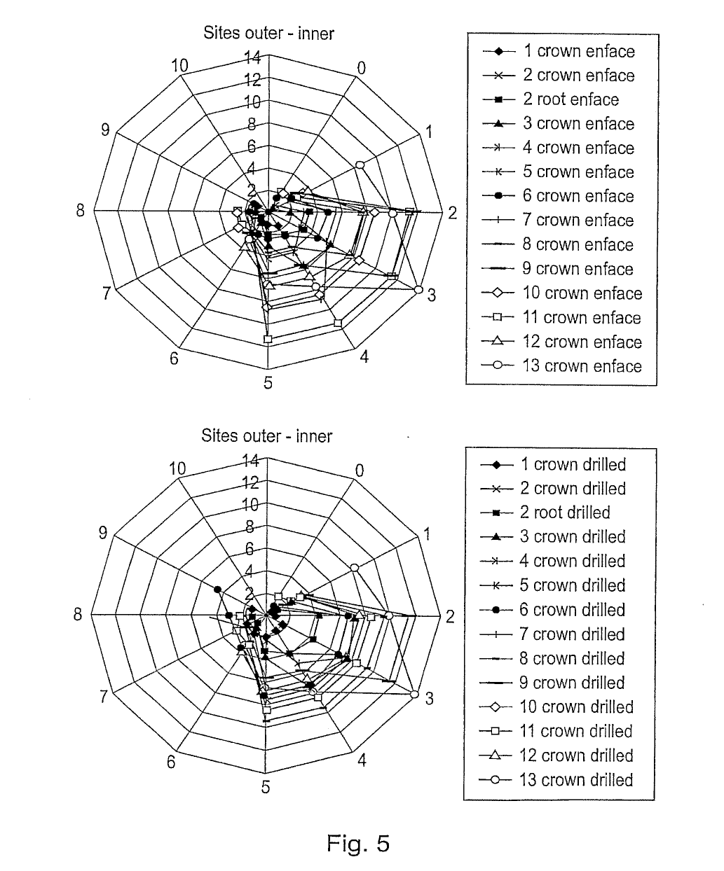

FIG. 5 shows en face and as drilled surface plots of decay samples;

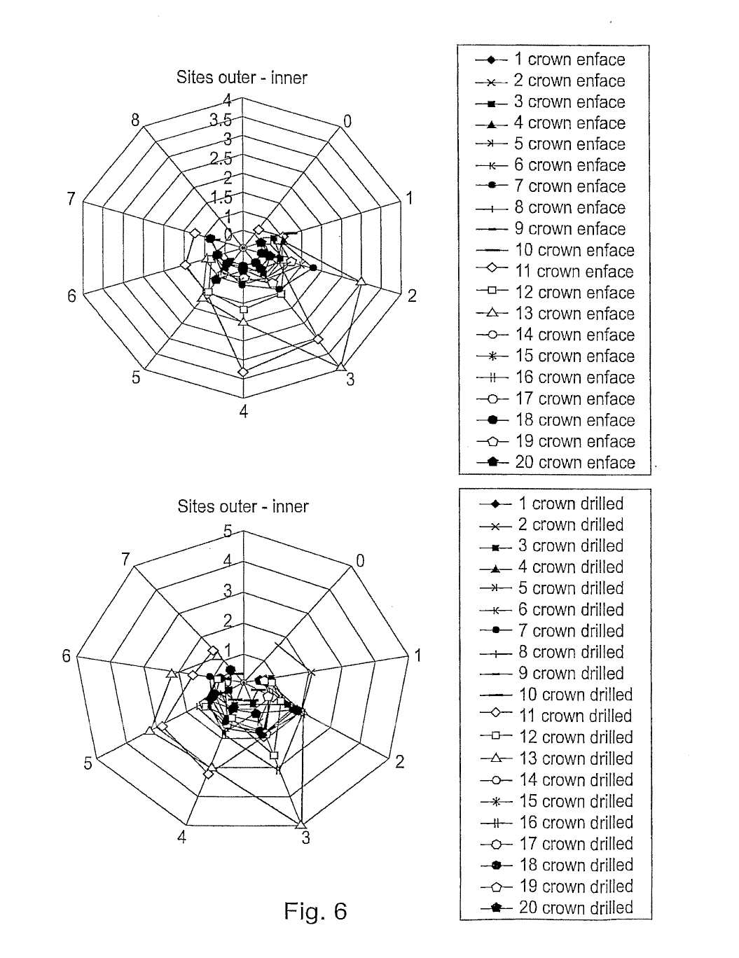

FIG. 6 shows en face and as drilled surface plots of control samples;

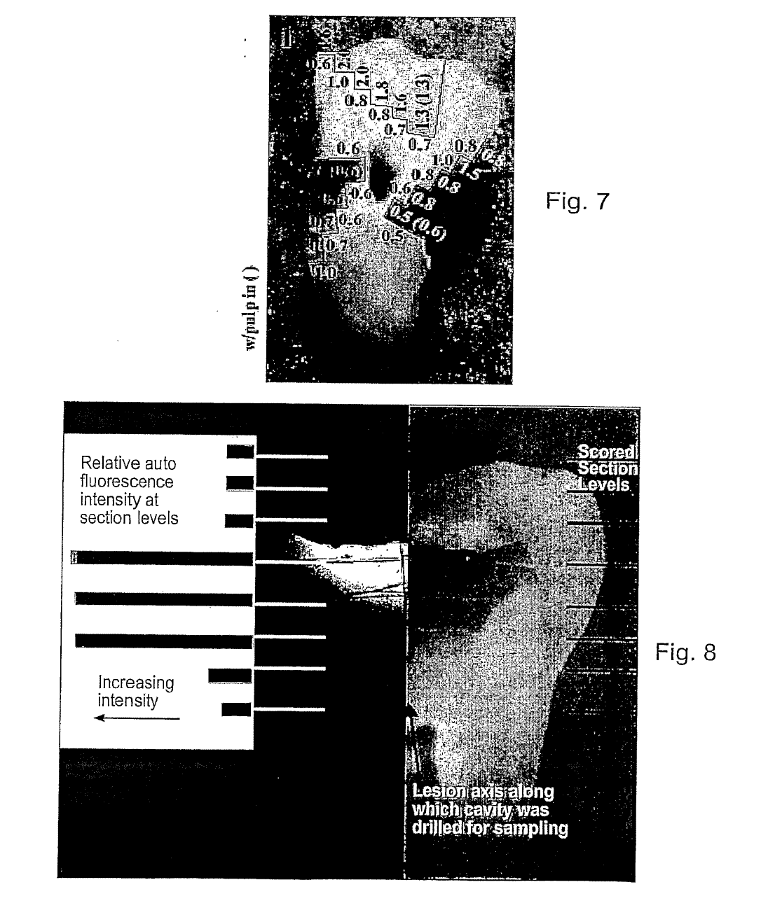

FIG. 7 shows cut steps in a control tooth and

FIG. 8 is a composite image showing a gross hemisected tooth decay lesion on the right with its mirror imaged gross auto-fluoresence signature on the left.

FIG. 1 schematically shows a first embodiment of an apparatus illustrating the present invention. The apparatus A comprises a light source B such as a laser source for producing a light beam C. An optical element D, in this example an optical fibre D has an input end optically coupled to the light source B to receive the light beam C and an output end arranged to direct the light beam as a confocal beam into tissue, in this example a tooth E. A detector F optically coupled to the optical element D receives a return beam G from the tooth stimulated by the confocal beam and generates an output dependent upon the return beam. In this example the beam G passes back along the optical fibre D to be received by detector F. The detector is connected to an analyser H which may be an oscilloscope for example, to provide an indication as to whether the confocal beam is directed onto infected tissue within the tooth.

FIG. 2 schematically shows a second, more detailed, embodiment of an apparatus illustrating the present invention. In this embodiment a source of electromagnetic radiation 1 is provided. A source of any electromagnetic radiation may be provided as long as it can be used to produce a confocal beam, such as a laser beam or an incoherent light source beam. For example, the electromagnetic source may be a source ranging from the visual to infra-red portions of the spectrum in conjunction with a suitable detector. In the example the source of electromagnetic radiation 1 is a 488 nm blue Argon ion laser. In this example the laser 1 directs a laser beam against a beam guide 2 such as a front surface reflector and into the instrument interstices. In this example the beam reflected by the beam guide 2 passes through an encoded spinning disk 3 for subtraction of ambient light as will be described later. The beam then encounters a beam guide 4 with a dichroic—long pass for yellow and greater wavelengths. The beam is then directed into a focussing objective 5 to introduce the beam into an optical fibre 6. In this example the optical fibre 6 is a bundle of coherent fibres of approximately 10 m in length acting as confocal apertures and wave guides. The other end of the optical fibre 6 directs the beam onto a tissue sample which in this example is a dentine sample 7 with a decay patch. The beam reflected by the tissue sample is directed back through the optical fibre 6 and the beam guide 4 to be received by a photodiode detector 8. In this example the detector 8 has an additional >570 nm long pass filter fitted to its sampling aperture. The detector is connected to an oscilloscope 9 to display a voltage received from the detector 8. In tests this apparatus was found to have a power at the tip of the optical fibre 6 of approximately 1 mW.

In this example subtraction of ambient light is performed by a so-called ‘lock in detection technique’. The encoded disk 3 is arranged to spin so that when it is open to the laser beam, the detector 8 receives ambient plus confocal excitation photons—but when closed to the laser beam, only ambient light is detected. Subtraction of the level of ambient light from the combined signals (differentiated in time by encoding of slit wheel shaft) leaves the confocal excitation voltage only remaining for display.

Examples of experimental methods to illustrate the advantages of the invention will now be described.

13 sections were created using a diamond wheel saw from a series of freshly extracted teeth, each with clinically obvious dental decay (with moisture maintained by normal saline immersion and no aldehyde/alcoholic cleansers). Each was sectioned through the centre of the decay—either via the crown or the root surface.

Sectioned carious surfaces were scored with a scalpel blade, a single axial line scored from surface to nerve space through a lesion and a series of “parallel” interval lines approximately 500 μm apart providing level lines throughout the depth of the lesion.

Measurements were taken using the apparatus described in FIG. 2 and directing the beam in two perpendicular planes—

1) en face—in which the tip of the optical fibre 6 was placed onto a sectioned dentine surface prior to drilling and

2) As drilled—in which a 1 mm diameter dental bur was used to cut a slot/channel by eye along one side of the axial lesion score line (as a clinical dentist would do in practice in a whole tooth); —stopping at each depth plane as indicated by the score levels described (levels 0-8). Autofluorescence measurements were taken at each plane to correspond to the en face measurements described. The drilled cavity was restricted to one side of the axial score line only.

For comparison and data corroboration/lesion co-localisation purposes, a gross anatomical image was matched with a composite frame of confocal autofluorescence signal (488 nm illumination/>540 nm long pass) (×5/0.2 na dry lens) for each lesion examined as shown in FIG. 3.

FIG. 3 shows matched images of two sectioned dentine decay lesions showing the axial drilling plane score line and transverse lesion level lines—numbered on each image. In the lower panels of FIG. 3 the corresponding bench microscope autofluorescence image of the decayed lesions is shown, clearly identifying the score lines for measurement location.

After drilling was completed, the remaining half of the lesion was clinically examined with a traditional dental probe to identify/confirm the position of the hard tissue/soft decay interface in each sample.

A control sample was also provided. A selection of sound teeth extracted for orthodontic purposes, was sectioned in an identical fashion and kept in identical conditions to the decayed samples described above. A series of 20 stepped cavities were cut—as a dental surgeon would drill into a tooth on the cut surfaces, with steps being at identical 0.5 mm intervals. Identical en face and drilled surface autofluorescence measurements were taken from each sample to act as sound dentine controls.

Published data shows that the autofluorescence signature of sound dentine intensifies if heated—a likely phenomenon at the very depths of the cavities drilled—
Matsumoto H, Kitamura S, Araki T. Applications of fluorescence microscopy to studies of dental hard tissue. Front Med Biol Eng. 2001; 10(4):269-84.
Matsumoto H, Kitamura S, Araki T. Autofluorescence in human dentine in relation to age, tooth type and temperature measured by nanosecond time-resolved fluorescence microscopy. Arch Oral Biol. 1999 April; 44(4):309-18.

Thus unexpected fluorescence rises detected by this instrument within the sound depth of a cavity can also be used to warn of or demonstrate thermal “abuse” of the dentine at the base of a cavity.

Furthermore, the fluorescence signature from each of the deepest (most pulpal) dentine steps was repeated with the extracted tooth nerve (pulp) tissue specimens in place and removed, to rule out additional contributions to the fluorescence signature from the adjacent pulpal tissue within deep cavities.

As absolute autofluorescence signatures of decayed and sound tissue inevitably vary between individuals and to a lesser extent between teeth/lesions of an individual, the user is looking for a significant drop in autofluorescence signature to show loss of decay related autofluorescence emission on completion of decay removal.

To confirm the autofluorescence signature was dependant on bacterial infection and dentine degradation, autofluorescence signatures and fluorescence lifetime imaging was undertaken on sound and phosphoric acid demineralised dentine samples. Lifetime and autofluorescence behaviours were indistinguishable in the two sample types, confirming a bacterial infection element was responsible for the autofluorescence signature being detected. Lifetimes have been shown to significantly alter in bacterially infected dentine (decay) as described in “Time-correlated single-photon counting fluorescence lifetime confocal imaging of decayed & sound dental structures with a white-light supercontinuum source” [McCONNELL, G.; GIRKIN, J. M.; AMEER-BEG, S. M.; BARBER, P. R.; VOJNOVIC, B.; N G, T.; BANERJEE, A.; WATSON, T. F; COOK, R. J. Journal of Microscopy, 225, (2) February 2007, pp. 126-136].

The results obtained are considered below. Peak autofluorescence emission spectra at 488 nm illumination, for both sound and decayed dentine, for one gross lesion, were taken at the time of overall lesion autofluorecence mapping, using the bench microscope. Sampling was undertaken at the centre of the softened decay lesion by visible autofluorescence signal and again for comparison at a remote site of uninvolved dentine as shown in FIG. 4. As can be seen, the fluoresence from the decayed part of the tooth is much stronger than from the healthy part. In fact, a more than 10 fold increase in autofluorescence signature was detected, peaking at 570-580 nm on 488 nm excitation (>570 nm long pass detection filtration).

Pure autofluorescence emission intensity data signals for each measurement site and orientation were recorded as a voltage output from the photodiode detector via the oscilloscope, with the ambient light contribution to each output signal having been eliminated as described above.

Data for each tooth sample was plotted as a voltage against plane position, centred around the hard/soft clinical interface and compared and the data presented as shown in FIG. 5.

Comparison of data from different lesions is problematic as not all decay sites were of the same depth, yielding varying numbers of “steps” within each lesion.

Thus for demonstration purposes, fluorescence intensity data are best plotted on a web diagram, intensity increasing from the centre and radial spokes identifying sampling steps. In all cases involving decay, the space between radial spokes 5-6 represent the hard-soft decayed dentine interface. Further, the Enamel-dentine junction interface is universally sited between spokes 1 & 2.

Graphical plots of both en face and drilled surfaces are compared in two separate graphs for the carious lesions in FIG. 5. The similarity of fluorescence peaks between first and fifth spokes reflect peak decayed dentine fluorescence within the lesions. Sharp cut-offs beyond point 5 demonstrate the loss of fluorescence signature as the decay is completely removed.

Concordance of the loss of autofluorescence and the change in clinical hardness of residual dentine is well accepted, and was very accurately detected in the apparatus used as shown in FIG. 2.

For comparison, the 20 control samples with no decay are again all centred on the Enamel-Dentine junction—located between spokes 1 and 2 as shown in FIG. 6. Cavity depths are variable—dictated by the size of each sample, but the majority of plots remain below the 2 volt limit compared to the majority of decay plots exceeding the 4 volt thresholds in the example of FIG. 5.

Comparison of the en face and drilled plots for each caries/control group show identical trends and patterns. In the control groups, only two samples breached the 2 volt line in the en face orientation. Likewise the same two samples and two others breach the 2 volt line in the drilled cavity group. These specimens showed unexpected decay deeper within the sample, not immediately apparent on first sectioning, but detected by the confocal probe more accurately than the eye.

The deepest cut dentine floors were all within 500 microns of the pulp (nerve) space. Concern existed that the pulp tissue may contribute to the fluorescence signature detected from the deepest reaches of the cavity.

The image of FIG. 7 shows cut steps in a control tooth. En face and cut surface voltages are shown as numbers superimposed on the image. Figures in brackets show the voltage detected with the residual nerve tissue in place in the pulp chamber.

Comparison of base dentine voltage+/−pulp tissue for 17 sites is shown in the table below. A common mean voltage of 1.1 v was noted with or without the nerve tissue with +/−0.6 v standard deviation. —ie no significant difference.

| | Without pulp | With pulp |
|---|---|---|
| mean voltage | 1.1 | 1.06 |
| recorded +/− pulp | | |
| SD | 0.58 | 0.61 |

Differences across interfaces are described numerically below:—

### Carious Samples:—

Overlying Enamel into Decayed Dentine at the Enamel Dentine Junction:

Drilled

| | Enamel | Decay dentine |
|---|---|---|
| Mean voltages | 2.57 | 6.94 |
| across EDJ | | |
| SD for mean | 2.02 | 3.02 |

En face

| | Enamel | Decay dentine |
|---|---|---|
| Mean voltages | 2.43 | 6.7 |
| across EDJ | | |
| SD for mean | 2.08 | 3.30 |

### Carious Samples:—

Decayed Soft Dentine into Sound Hard Dentine Interface:

### Significant Fall in Fluorescence Signature Once Returned to Deeper Harder Dentine:—

Drilled

| | Decay dentine | Deeper hard dentine |
|---|---|---|
| Mean voltage change | 5.11 | 2.16 |
| across interfaces | | |
| SD for mean | 2.50 | 0.87 |

En face

| | Decay dentine | Deeper hard dentine |
|---|---|---|
| Mean voltage change | 4.91 | 2.14 |
| across interfaces | | |
| SD for mean | 3.08 | 0.87 |

Voltage detected halves across the interface

### Control Samples

### Sound Enamel Dentine Junctions—

### No Significant Autofluorescence Signature Rise Across the Interface:—

Drilled

| | Enamel | Dentine |
|---|---|---|
| Mean voltages | 0.68 | 1.24 |
| across EDJ | | |
| SD for mean | 0.35 | 0.48 |

En face

| | Enamel | Dentine |
|---|---|---|
| Mean voltages | 0.72 | 1.07 |
| across EDJ | | |
| SD for mean | 0.21 | 0.56 |

### Bulk Dentine Fluoresence Voltage—Means of all Sound Dentine Measurements Taken, Whatever the Depth—

En face

| | |
|---|---|
| Mean voltage | 0.91 |
| SD | 0.57 |

Drilled

| | |
|---|---|
| Mean voltage | 1.20 |
| SD | 0.72 |

The small (but not significant) rise may reflect a thermal effect in the drilled group. A whole result trend showed 4 cases where the drilled group showed a secondary increase in dentine fluorescence in the cavity depths—some samples were difficult to cool at extreme depth and occasional warming of the dentine is a very likely explanation. Although occurring occasionally in practice—it is unusual to drill so deep and narrow a channel into a tooth. The coolant access being far more efficient in larger cavities.

A single summary image is presented as FIG. 8. FIG. 8 shows a composite image showing a gross hemisected tooth decay lesion on the right, with its mirror imaged gross auto-fluorescence signature on the left. The relatively thin horizontal lines represent the confocal micro-probe sampling planes, centred along the vertical mid-lesion axis score mark. The relative fluorescence intensities are shown as a relatively thick horizontal line bar chart to the left of the image.

The apparatus of embodiments of the present invention including a confocal optical probe allows thin film depths of dental caries to be detected by sampling the autofluorescence from only a shallow depth of tissue under examination.

Elimination of bulk background signals thus eliminates the “swamping” effect, thus markedly increasing the sensitivity of this residual decay detection system.

The data shows clear drop off in fluorescence beyond the soft-hard decayed dentine interface as judged clinically as expected and identified in laboratory based sectioned surface bench confocal microscope studies:—
Banerjee A. (1998) Applications of scanning microscopy in the assessment of Dentine Caries and methods of its removal. PhD Thesis, U. of London.
Banerjee A., Boyde A. (1998). Autofluorescence and mineral content of carious dentine: scanning optical and backscattered electron microscopic studies. Caries Res. 32, 219-226.
Banerjee A. et al. (1999) A confocal microscopic study relating the autofluorescence of carious dentine to its microhardness. Brit. Dent. J. 187, 206-210.
Banerjee A. et al. (2003) In vitro validation of carious dentin removed using different excavation criteria. Amer. J. Dent. 16, 228-230.

Further, as expected because decay spreads laterally at the enamel-dentine junction (EDJ), a sharp rise in fluorescence data was recorded across the EDJ, into the softened decayed dentine.

A confocal fibre optic residual caries detector of an embodiment of the present invention thus offers substantial advantages in the discrimination of thin layers of residual decay in the base of dentine cavities.

An additional benefit of increased signature fluorescence on thermal results may also offer additional clinical advantages in a system incorporating an embodiment of the present invention, warning of likely increased sensitivity in the post operative period and possible damage to the underlying pulp tissue. This may be used to direct therapy towards sedative (temporary) linings in deep cavities.
Matsumoto H, Kitamura S, Araki T. Autofluorescence in human dentine in relation to age, tooth type and temperature measured by nanosecond time-resolved fluorescence microscopy. Arch Oral Biol. 1999 April; 44(4):309-18.

Such information is likely only to be discriminated by an optical sampling system that differentiates between local (subjacent) tissue to the sampling site, while avoiding the overwhelming bulk autofluorescence signature from the remaining tooth as in an embodiment of the present invention.

Thus confocal small volume autofluorescence offers significant improvements over non confocal bulk sampling systems by being able to define residual thin films of decay (and thermal damage) providing valuable clinical data concerning drilling end points and potential thermal induced additional pulpal damage.

1. An apparatus for detecting infected tissue, the apparatus comprising a light source for producing a light beam; an optical element having an input end optically coupled to the light source and an output end arranged to direct the light beam as a confocal beam into tissue; a detector optically coupled to the optical element to receive a return beam back from the tissue stimulated by the confocal beam and to generate an output dependent upon the return beam and an analyser for analysing the output of the detector to determine whether the return beam is indicative of infected tissue.

2. The apparatus according to claim 1, wherein the optical element is arranged to direct the confocal beam to a predetermined point or depth in the tissue.

3. The apparatus of claim 1, wherein the apparatus is arranged to vary the predetermined point or depth in the tissue at which the confocal beam is directed.

4. The apparatus according to claim 1, wherein the analyser is arranged to reduce the effect of ambient light.

5. The apparatus according to claim 4, wherein the analyser is arranged to reduce the effect of ambient light by subtracting an output indicative of just ambient light from an output indicative of both ambient light and a return beam from the tissue stimulated by the confocal beam.

6. The apparatus according to claim 1, wherein the light source is a laser light source.

7. The apparatus according to claim 1, wherein the optical element is an optical fibre.

8. The apparatus according to claim 1, including an indicator to indicate the presence or absence of infected tissue.

9. A dental device arranged to receive a control signal from an apparatus according to claim 1.

10. A method of detecting infected tissue, the method comprising producing a light beam; directing the light beam into an input end of an optical element having an output end arranged to direct the light beam as a confocal beam into tissue; detecting a return beam back from the tissue stimulated by the confocal beam; generating an output dependent upon the detected return beam and analysing the generated output to determine whether the return beam is indicative of infected tissue.

11. A method according to claim 10, wherein the confocal beam is directed to a predetermined point or depth in the tissue.

12. A method according to claim 11, wherein the confocal beam is directed to a plurality of points or depths in the tissue.

13. A method according to claim 10, wherein the generated output is used to control a dental cutting device.

14. A method according to claim 10, wherein the confocal beam is directed into a tooth.

15. An apparatus substantially as hereinbefore described with reference to the accompanying drawings.

16. A method substantially as herein before described with reference to the accompanying drawings.
